SEQUENCE LISTING (6 sequences)

SEQ ID NO: 1 - DNA, 22 bp, Artificial Sequence
  Primer
catgcacagctcagcactgctc

SEQ ID NO: 2 - DNA, 17 bp, Artificial Sequence
  Primer
gtcgccaccctgatgtc

SEQ ID NO: 3 - DNA, 24 bp, Artificial Sequence
  Primer
gactactatgctaaagaggtcacc

SEQ ID NO: 4 - DNA, 24 bp, Artificial Sequence
  Primer
ctgatcccgttgatttccacgtgg

SEQ ID NO: 5 - DNA, 21 bp, Artificial Sequence
  Primer
ccagtatgactccactcacgg

SEQ ID NO: 6 - DNA, 22 bp, Artificial Sequence
  Primer
caacctggtcctcagtgtagcc

(19) United States
(12) Patent Application Publication
(10) Pub. No.: US 2004/0071677 A1
(43) Pub. Date: Apr. 15, 2004

(54) Nephritis treatment by gene therapy

(76) Inventors: Jong-Gu Park, Daegu (KR); Young-Kook Choi, Daegu (KR)

Correspondence Address: JHK Law, P.O. Box 1078, La Canada, CA, 91012-1078, US

(21) Appl. No.: 10/634,408

(22) Filed: Aug. 5, 2003

Related U.S. Application Data
(60) Provisional application No. 60/401,277, filed on Aug. 5, 2002.

(51) Int. Cl. A61K 48/00

(52) U.S. Cl. 424/93.21; 514/44; 424/93.2

(57) ABSTRACT

The present application discloses a method of reducing inflammation in kidney of a subject, and includes treating nephritis and glomerulosclerosis, which method includes delivering to the kidney of the subject in need thereof a therapeutically effective amount of a gene encoding an anti-inflammatory or immunosuppressant protein. Gene encoding IL-10 is delivered to the kidney.

### CROSS-REFERENCE TO RELATED APPLICATIONS

The present application claims the benefit of priority to U.S. Provisional Application No. 60/401,277, filed Aug. 5, 2002, which is incorporated by reference herein in its entirety.

### BACKGROUND OF THE INVENTION

1. Field of the Invention

The present invention relates to a method of introducing at least one gene encoding an anti-inflammatory or immunosuppressant protein into at least one mammalian renal cell for use in treating or preventing nephritis and/or proteinuria in the mammalian host.

2. General Background and State of the Art

Nephritis, or inflammation of the kidneys, generally is caused by an infection. Signs of nephritis, including blood and protein in the urine and impaired kidney function, depend on the type, location, and intensity of the immune reaction. Nonetheless, the many different conditions that injure the kidneys can produce similar types of damage, symptoms, and outcomes. Generally, inflammation does not affect the entire kidney. The resulting disease depends on whether the inflammation affects primarily the glomeruli, the tubules and the tissues that surround them (tubulointerstitial tissue), or the blood vessels within the kidneys.

Glomerulosclerosis is characterized by sclerosis or scar formation of the capillary tuft of glomeruli. Focal Segmental Glomerulosclerosis (FSGS), the primary glomerular disease of glomerulosclerosis, brings about heavy proteinuria and renal insufficiency (Kamanna VS et al., Histol Histopathol 13: 169-179 (1998)). Secondary glomerulosclerosis is detected in various conditions including almost any kind of end stage renal disease. The proposed pathogenesis of glomerulosclerosis is podocyte injury or glomerular hyperfiltration by insufficient amounts of nephrons. Although the mechanism underlying the disease progression remains unanswered, the activity of interstitial infiltrating lymphocytes is strongly suspected for disease progression (Del PD et al., Nephrol Dial Transplant 13 (Suppl.): 20-25 (1998). Large amounts of proteins that are leaked from injured glomeruli are reabsorbed by the proximal tubular epithelium. The proteins are modified in lysosomes, which then attain a chemotactic function for circulating lymphocytes and macrophages (Bogenschuz O et al., Am J Nephrol 10: 137-147 (1990)). The cytokines released by these recruited inflammatory cells can damage both the tubular epithelial cells and surrounding vessels. This damage, in turn, disconnects linkages to glomeruli and makes them isolated. Repeated cycles of this type of damage are responsible for further deterioration of glomerulosclerosis and worsening renal functions (Wehrmann M et al., Clin Nephrol 33: 115-122 (1990); Mackensen-Haen S et al., Clin Nephrol 37: 70-77 (1992); Bohle A et al., Kidney Int 67 (Suppl.): 186S-188S (1998)).

Current therapeutic methods for glomerulosclerosis are not satisfactory because long-term exposure to conventional drugs induces side effects to several organs. Further, the drugs do not necessarily interrupt or reverse progression of the disease, and thus kidney dialysis or transplantation is required to manage end-stage renal failure. Modification of an inflammatory response in affected glomeruli could attenuate the pathological consequences of kidney failure as inflammation is a salient feature of the disease and as the disease has a convincing immunologic background. Therefore, it seems reasonable that intervention in the progression of the disease to prevent kidney failure is possible by bolstering anti-inflammatory mediators using anti-inflammatory cytokines or blocking the production of proinflammatory mediators (Kitching AR et al., Kidney Int 52: 52-59 (1997); Rui-Mei L et al., Kidney Int 53: 845-852 (1998)).

IL-10, originally termed cytokine synthesis inhibitory factor, is a 35 kD homodimeric cytokine which is produced by macrophages, Type 2 Helper T (Th2) cells, and B cells (Fiorentino DF et al., J Exp Med 170: 2081-2095 (1989)). IL-10 participates mainly in immune suppressive roles which are mediated through the inhibition of proinflammatory cytokines produced by Th1 cells (De Wall MR et al., J Exp Med 174: 915-924 (1991)) and the down regulation of antigen presentation by dendritic cells and macrophages (Buelens C et al., Eur J Immunol 25: 2668-2672 (1995); Igietseme JU et al., J Immunol 164: 4212-4219 (2000)).

In experimental animal models of nephritis, nephritis can be induced by high-protein diet, chemical reagents such as puromycin, and unilateral ureteric obstruction (Irwin K C et al., Pediatr Nephrol 6: 448-450 (1992); Korzets Z et al., Nephrol Dial Transplant 12: 286-288 (1997); Garber S L et al., Am J of Kidney Dis 33: 1033-1039 (1999); Taal M W et al., Kidney Int 58: 1664-1676 (2000); Ayan S et al., Urology 58: 301-306 (2001); Chen H C et al., J Lab Clin 137: 279-283 (2001); Garber S L et al., Kidney Int 59: 876-882 (2001). In the FGS/Kist mouse strain, FSGS that eventually progresses into kidney failure with nephritis develops spontaneously followed by tubular atrophy and fibrosis of glomeruli as well as interstitium. The mice also exhibit progressive aggravation of proteinuria with aging. Proteinuria develops about 40 days after birth in the mice, and kidney failure within one year (Hyun B H et al., Lab Anim Sci 35: 519-526 (1991)).

There is a continuous need in the art for a better method of treating or preventing nephritis.

### SUMMARY OF THE INVENTION

The invention overcomes the above-mentioned problems, and provides a method of treating or preventing development of nephritis and proteinuria by expressing an immunosuppressive and/or anti-inflammatory protein in the kidney.

The present invention is directed to a method of reducing inflammation in kidney of a subject, comprising delivering to the kidney of the subject in need thereof a therapeutically effective amount of a gene encoding an anti-inflammatory or immunosuppressant protein. Preferably, the gene may encode IL-1Ra, IL-4, IL-6, IL-10, IL-16, or TGF-β1. In particular, the gene may encode IL-10. In the above method, the gene is inserted into a vector. Preferably, the vector may be a virus. And in particular, the vector may be an adenovirus or an adeno-associated virus or retrovirus. Alternatively, the vector may be a plasmid.

In the above method, the gene may be transfected into a population of cells in vitro, wherein the transfected population of cells is administered to the subject. In such a method, the gene may preferably encode IL-1Ra, IL-4, IL-6, IL-10, IL-16, or TGF-β1. In particular, the gene may encode IL-10.

The present application is also directed to a method of treating nephritis comprising delivering to the kidney of the subject in need thereof a therapeutically effective amount of a gene encoding an anti-inflammatory or immunosuppressant protein. In particular, the nephritis may be glomerulopathy. Further in particular, the glomerulopathy may be glomerulosclerosis. Preferably, the gene may encode IL-1Ra, IL-4, IL-6, IL-10, IL-16, or TGF-β1. In particular, the gene may encode IL-10.

The present application is also directed to a method of preventing nephritis in a patient predisposed to such a condition, comprising delivering to the kidney of the subject in need thereof a therapeutically effective amount of a gene encoding an anti-inflammatory or immunosuppressant protein. In particular, the nephritis may be glomerulosclerosis. Preferably, the gene may encode IL-1Ra, IL-4, IL-6, IL-10, IL-16, or TGF-β1. In particular, the gene may encode IL-10.

The present application is also directed to a method of reducing excretion of polypeptides in urine of a subject suffering from a renal disorder comprising delivering to the kidney of the subject in need thereof a therapeutically effective amount of a gene encoding an anti-inflammatory or immunosuppressant protein. Preferably, the gene encodes IL-1Ra, IL-4, IL-6, IL-10, IL-16, or TGF-β1. In particular, the gene encodes IL-10.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGURES 1A-1E show β-galactosidase expression in the kidney of FGS/Kist mice transduced with Ad:LacZ. The left kidney of FGS/Kist mice was transduced with 20 μl of Ad:LacZ (5×10⁹ p.f.u./ml). After sacrifice of the transduced mice, kidneys were fixed by glutaraldehyde perfusion and incubated in X-gal solution (1.5 mM potassium ferricyanide, 1.5 mM potassium ferrocyanide and 1% X-gal) for 4 h and then examined under a microscope (A-C). The kidneys were examined for β-galactosidase expression by histochemical analysis of paraffin sections of the kidney (D and E). Sections of 4 μm were counterstained with eosin and hematoxylin. (A) Sham, (B) The left kidney transduced with Ad:LacZ, (C) Contralateral (Right) kidney, (D) Sham, (E) The left kidney transduced with Ad:LacZ. (A-C: original magnification 20×, D and E: original magnification 400×)

FIGS. 2A-2C show β-galactosidase expression in the kidneys and other organs of FGS/Kist mice. The whole kidney transduced with Ad:LacZ locally (B) and intravenously (C) was homogenized in an ice-cold buffer of 0.25 M Tris-HCl, pH 8.0 at each time point. The homogenate was frozen and thawed 3 times and then centrifuged at 3000 rpm for 5 min. β-galactosidase activity in the extract was measured at an O.D. of 420 nm and normalized to a total protein content. LacZ expression was also analyzed for the heart, the spleen, the lung and the liver. (A) ▪: Left kidney, □: Right kidney, (B) β-galactosidase expression in the liver (●), the spleen (▴), the lung (◯), and the heart (⋄) through parenchymal injection, (C) β-galactosidase expression in the liver (●), the spleen (▾), the lung (◯), the kidney (□), and the heart (⋄), through intravenous injection. Each value represents the mean±s. d. values (n=3).

FIGS. 3A-3B show in situ RT-PCR on tissue sections from FGS/Kist mice transduced with Ad:hIL-10. Oligonucleotides hybridizing to human IL-10 cDNA were used for in situ RT-PCR that was carried out with dNTPs containing digoxigenin UTP. IL-10 RT-PCR products labeled with digoxigenin were incubated with anti-digoxigenin antibodies conjugated with alkaline phosphatase and color reaction was performed using NBT/BCIP. (A) Kidney transduced with Ad:LacZ. No signal was detected. (B) Kidney transduced with Ad:hIL-10. The NBT/BCIP color reaction yielded brown staining (no counterstaining). (200×)

FIGS. 4A-4D show histological analyses of glomeruli and tubules from FGS/Kist mice after Ad:LacZ or Ad:hIL-10 transduction. Four weeks (at the age of 10 weeks) post injection of 20 μl Ad:hIL-10 (5×10⁹ p.f.u./ml) in the left kidney, kidneys were harvested, sectioned and stained with PAS. (A) Kidney at 5 weeks (200×). Glomeruli are intact and the interstitium is devoid of the infiltration of inflammatory cells. (B) Kidney at 10 weeks (200×). Glomeruli are segmentally sclerotic and lymphocytes denoted by an open arrow are found to be infiltrated in the interstitium. (C) Kidney at 10 weeks after transduction with Ad:LacZ (100×). Glomerulosclerosis is shown with tubular atrophy with plugged casts, which are denoted by a closed arrow. (D) Kidney at 10 weeks after transduction with Ad:hIL-10 (100×). Glomeruli are well preserved and devoid of the infiltration of inflammatory cells.

FIG. 5 shows quantitative RT-PCR analysis of TGF-β1 mRNA in the kidney from FGS/Kist mice after Ad:LacZ or Ad:hIL-10 transduction. Results were expressed as the relative ratio of intra-kidney TGF-β1 to GAPDH mRNA. #, P<0.01 as compared with the naive group at 10 weeks or the Ad:LacZ treated group. Each value represents the mean±s. d. values (n=6).

FIGS. 6A-6E show detection of TGF-β1 expression in the kidney of FGS/kist mice. Cryostat sections were incubated with a mouse anti-TGF-β1 antibody for immunohistochemistry (A-E). Arrows indicate representative TGF-β1 positive cells. (A) The naive kidney at 6 weeks, (B) The naive kidney at 10 weeks, (C) The kidney transduced with Ad:LacZ, (D) The kidney transduced with Ad:hIL-10, and (E) The contralateral kidney of Ad:hIL-10 treated mice. (400×)

### DETAILED DESCRIPTION OF THE PREFERRED EMBODIMENTS

In the present application, “a” and “an” are used to refer to both single and a plurality of objects.

As used herein, “acute nephritic syndrome” or “acute glomerulonephritis” refers to an inflammation of the glomeruli that results in the sudden appearance of blood in the urine, with clumps of red blood cells (casts) and variable amounts of protein in the urine. Acute nephritic syndrome may follow a streptococcal infection, such as strep throat. The glomeruli are damaged by the accumulation of antigen from the dead streptococci clumped together with the antibodies that neutralize them. These clumps (immune complexes) coat the membranes of the glomeruli and interfere with their filtering function. Acute nephritic syndrome may also be caused by a reaction to other infections, such as infection of an artificial body part (prosthesis), bacterial endocarditis, pneumonia, abscesses of abdominal organs, chickenpox, infectious hepatitis, syphilis, and malaria. The last three infections may cause nephrotic syndrome rather than acute nephritic syndrome.

As used herein, “anti-inflammatory” protein refers to any protein that inhibits local immune responses. Such immune responses include vasodilatation, leukocyte infiltration, redness, heat, activation of local cells and immune cells, and the release of inflammatory mediators from these cells. It is to be understood that certain proteins may act as a pro-inflammatory agent, and later become anti-inflammatory depending on the cellular environment. Further, “anti-inflammatory” and “immunosuppressant” or “immunosuppressive” protein typically refers to a cytokine that affects the diseased area by suppressing or countering immunologic and/or pro-inflammatory activities. Such cytokines may include, without limitation IL-1Ra, IL-4, IL-6, IL-10, IL-16, or TGF-β1. It is also to be understood that by any particular gene/protein, the invention encompasses the gene/protein and any obvious variants thereby, which may be allelic variants or other modifications, which maintain the immunosuppressive and/or anti-inflammatory activities. The present invention also includes families of the gene/protein, which are related by sequence similarity or function.

As used herein, the term “biologically active” used in relation to liposomes denotes the ability to introduce functional DNA and/or proteins into the target cell.

As used herein, the term “biologically active” in reference to a nucleic acid, protein, protein fragment or derivative thereof is defined as an ability of the nucleic acid or amino acid sequence to mimic a known biological function elicited by the wild type form of the nucleic acid or protein.

As used herein, “chronic nephritic syndrome” or “chronic glomerulonephritis” refers to a disorder occurring in several diseases in which the glomeruli are damaged and kidney function degenerates over a period of years.

As used herein, “glomerulopathy” refers to kidney disorders in which inflammation affects mainly the glomeruli. There are four major types of glomerulopathies: (1) Acute nephritic syndrome, which starts suddenly and usually resolves quickly; (2) Rapidly progressive nephritic syndrome, which starts suddenly and worsens rapidly; (3) Nephrotic syndrome, which leads to the loss of large amounts of protein in the urine; and (4) Chronic nephritic syndrome, which starts gradually and worsens very slowly, often over a period of years. When the glomerulus is damaged, substances not normally filtered out of the bloodstream, such as proteins, blood, white blood cells, and debris, can pass through the glomerulus and enter the urine. Tiny blood clots (microthrombi) may form in the capillaries that supply the glomerulus.

As used herein, the term “mammalian host” includes members of the animal kingdom including, but not limited to, human beings.

As used herein, “nephritis-related disease” refers to primary glomerulopathies (acute diffuse proliferative glomerulonephritis, crescentic glomerulonephritis, membraneous glomerulopathy, lipoid nephrosis, focal segmental glomerulosclerosis, membranoproliferative glomerulonephritis, IgA nephropathy, focal proliferative glomerulonephritis, and chronic glomerulonephritis), systemic diseases (systemic lupus erythematosus, diabetes mellitus, amyloidosis, Goodpasture's syndrome, polyarteritis nodosa, Welgener's granulomatosis, Henoch-Schonlein purpura, and Bacterial endocarditis), and hereditary disorders (Alport's syndrome, and Fabry's disease).

As used herein, “nephrotic syndrome” refers to a collection of symptoms caused by many diseases that affect the kidneys, resulting in a severe, prolonged loss of protein into the urine, decreased blood levels of protein (especially albumin), retention of excess salt and water in the body, and increased levels of fats (lipids) in the blood. Nephrotic syndrome can be caused by any of the glomerulopathies or a vast array of diseases. Typically, the syndrome progresses to complete kidney failure in 3 or 4 months.

As used herein, “pharmaceutically acceptable carrier and/or diluent” includes any and all solvents, dispersion media, coatings, antibacterial and antifungal agents, isotonic and absorption delaying agents and the like. The use of such media and agents for pharmaceutically active, substances is well known in the art. Except insofar as any conventional media or agent is incompatible with the active ingredient, use thereof in the therapeutic compositions is contemplated. Supplementary active ingredients can also be incorporated into the compositions.

As used herein, a “promoter” can be any sequence of DNA that is active, and controls transcription in an eucaryotic cell. The promoter may be active in either or both eucaryotic and procaryotic cells. Preferably, the promoter is active in mammalian cells. The promoter may be constitutively expressed or inducible. Preferably, the promoter is inducible. Preferably, the promoter is inducible by an external stimulus. Preferably, the promoter is a viral promoter. Likewise, “enhancer elements”, which also control transcription, can be inserted into the DNA vector construct, and used with the construct of the present invention to enhance the expression of the gene of interest.

As used herein, “rapidly progressive nephritic syndrome” or “rapidly progressive glomerulonephritis” refers to disorder in which most of the glomeruli are partly destroyed, resulting in severe kidney failure with protein, blood, and red blood cell clumps (casts) in the urine.

As used herein, “selectable marker” includes a gene product that is expressed by a cell that stably maintains the introduced DNA, and causes the cell to express an altered phenotype such as morphological transformation, or an enzymatic activity. Isolation of cells that express a transfected gene is achieved by introduction into the same cells a second gene that encodes a selectable marker, such as one having an enzymatic activity that confers resistance to an antibiotic or other drug. Examples of selectable markers include, but are not limited to, thymidine kinase, dihydrofolate reductase, aminoglycoside phosphotransferase, which confers resistance to aminoglycoside antibiotics such as kanamycin, neomycin and geneticin, hygromycin B phosphotransferase, xanthine-guanine phosphoribosyl transferase, CAD (a single protein that possesses the first three enzymatic activities of de novo uridine biosynthesis—carbamyl phosphate synthetase, aspartate transcarbamylase and dihydroorotase), adenosine deaminase, and asparagine synthetase (Sambrook et al. Molecular Cloning, Chapter 16. 1989), incorporated herein by reference in its entirety.

Glomerulosclerosis

Glomerulosclerosis is characterized by the infiltration of circulating inflammatory cells, fibrosis of interstitium and tubular atrophy, and is shown in patients with advanced nephritis. Glomerular injury caused by several factors brings about proteinuria in which proteins bind with soluble immunoglobulin A (sIgA), sIgG and sIgM, forming immune complexes on the basement membrane. These immune complexes function as a chemotactic factor for inflammatory lymphocytes, which cause excessive immune responses in the affected areas (Bohle A et al., Kidney Int 67 (Suppl.): 186S-188S (1998)). When tubules are damaged by inflammatory cells, blood vessels connected with glomeruli are also injured and occluded. As a consequence, glomeruli become adversely affected and deteriorate. These glomerular changes are accompanied by tissue fibrosis and progress into eventual renal failure (Ratscchek M et al., Clin Nephrol 25: 221-226 (1986); Bohle A et al, Clin Nephrol 29: 28-34 (1998); Bohle A et al., Kidney Blood Press Res 19:191-195 (1996).

Focal Segmental Glomerulosclerosis (FSGS) is a common form of kidney disease that may cause permanent kidney failure in children and adults. Of children who have glomerulonephritis (inflammation of the kidney filtering units, or glomeruli) and nephrotic syndrome (large amounts of protein in the urine), 7 to 15 percent will be found to have focal glomerulosclerosis. Most adults with heavy protein loss in their urine have high blood pressure or diabetes, the leading causes of kidney disease. But some of these adults have no known cause of kidney disease.

Gene Therapy

The present invention discloses ex vivo and in vivo techniques for delivery of a DNA sequence of interest to the kidney tissue cells of the mammalian host. The ex vivo technique involves culture of target kidney tissue cells, in vitro transfection of the DNA sequence, DNA vector or other delivery vehicle of interest into the kidney tissue cells, followed by transplantation of the modified kidney tissue cells to the target kidney of the mammalian host, so as to effect in vivo expression of the gene product of interest.

It is to be understood that it is possible that substances such as a scaffolding or a framework as well as various extraneous tissues may be implanted together in the gene therapy protocol of the present invention. However, it is also possible that such scaffolding or tissue not be included in the injection system of the invention. In a preferred embodiment, in a cell-mediated gene therapy or somatic cell therapy, the invention is directed to a simple method of injecting a population of transfected or transduced tissue cells or the vector construct to the kidney so that the exogenous anti-inflammatory or immunosuppressant protein is expressed in the kidney. Injections may also be made via femoral vein or other accessible veins, which may allow the transfected cells or recombinant vectors to reach the kidneys in a more efficient manner.

As an alternative to the in vitro manipulation of host cells, the gene encoding the product of interest is introduced into liposomes and injected directly into the kidney area, where the liposomes fuse with the kidney tissue cells, resulting in in vivo gene expression of the anti-inflammatory or immunosuppressant gene product.

As an additional alternative to the in vitro manipulation of kidney tissue cells, the gene encoding the product of interest is introduced intraparenchymally as naked DNA. The naked DNA enters a kidney tissue cell, resulting in an in vivo gene expression of the gene product.

One ex vivo method of treating a kidney tissue disorder comprises initially generating a recombinant viral or plasmid vector which contains a DNA sequence encoding a protein or biologically active fragment thereof. This recombinant vector is then used to infect or transfect a population of in vitro cultured kidney cells, resulting in a population of kidney cells containing the vector. These kidney tissue cells are then transplanted to a target kidney of a mammalian host, effecting subsequent expression of the protein or protein fragment within the kidney. Expression of this DNA sequence of interest is useful in substantially reducing at least one deleterious kidney pathology associated with a kidney tissue disorder.

It will be understood by the artisan of ordinary skill that the preferred source of cells for treating a human patient is the patient's own kidney tissue cells, such as autologous kidney cells.

More specifically, this method includes employing as the gene a gene capable of encoding an immunosuppressive and/or anti-inflammatory protein, or a biologically active derivative or fragment thereof and a selectable marker, or a biologically active derivative or fragment thereof.

A further embodiment of the present invention includes employing as the gene a gene capable of encoding an immunosuppressive and/or anti-inflammatory protein or a biologically active derivative or fragment thereof, and employing as the DNA plasmid vector any DNA plasmid vector known to one of ordinary skill in the art capable of stable maintenance within the targeted cell or tissue upon delivery, regardless of the method of delivery utilized.

One such method is the direct delivery of the DNA vector molecule, whether it be a viral or plasmid DNA vector molecule, to the target cell or tissue. This method also includes employing as the gene a gene capable of encoding an immunosuppressive and/or anti-inflammatory protein or biologically active derivative or fragment thereof.

Another embodiment of this invention provides a method for introducing at least one gene encoding a product into at least one kidney cell for use in treating the mammalian host. This method includes employing non-viral means for introducing the gene coding for the product into the kidney tissue cell. More specifically, this method includes liposome encapsulation, surface complexation with cationic liposomes, complex formation with peptide or proteins that enhance transfection, calcium phosphate coprecipitation, electroporation, or DEAE-dextran mediation, and includes employing as the gene a gene capable of encoding an immunosuppressive and/or anti-inflammatory protein or biologically active derivative or fragment thereof, and a selectable marker, or biologically active derivative or fragment thereof.

Another embodiment of this invention provides an additional method for introducing at least one gene encoding a product into at least one cell of a kidney tissue for use in treating the mammalian host. This additional method includes employing the biologic means of utilizing a virus to deliver the DNA vector molecule to the target cell or tissue. Preferably, the virus is a pseudo-virus, the genome having been altered such that the pseudovirus is capable only of delivery and stable maintenance within the target cell, but not retaining an ability to replicate within the target cell or tissue. The altered viral genome is further manipulated by recombinant DNA techniques such that the viral genome acts as a DNA vector molecule which contains the heterologous gene of interest to be expressed within the target cell or tissue.

A preferred embodiment of the invention is a method of delivering an immunosuppressive and/or anti-inflammatory protein to a target kidney by delivering the immunosuppressive and/or anti-inflammatory protein gene to the kidney tissue of a mammalian host through use of an adenoviral or a retroviral vector with the ex vivo technique disclosed within this specification. In other words, a DNA sequence of interest encoding a functional adenoviral or immunosuppressive and/or anti-inflammatory protein or protein fragment is subcloned into an adenoviral or retroviral vector of choice, the recombinant viral vector is then grown to adequate titer and used to infect in vitro cultured kidney tissue cells, and the transduced kidney tissue cells, preferably autografted cells, are transplanted into the kidney of interest, preferably by injection.

Another preferred method of the present invention involves direct in vivo delivery of an immunosuppressive and/or anti-inflammatory protein gene to the kidney tissue of a mammalian host through use of either an adenovirus vector, adeno-associated virus (AAV) vector or herpes-simplex virus (HSV) vector. In other words, a DNA sequence of interest encoding a functional immunosuppressive and/or anti-inflammatory protein or protein fragment is subcloned into the respective viral vector. The immunosuppressive and/or anti-inflammatory protein containing viral vector is then grown to adequate titer and directed into the kidney, preferably by injection.

Direct intraparenchymal injection of a DNA molecule containing the gene of interest into the kidney results in transfection of the recipient kidney tissue cells and hence bypasses the requirement of removal, in vitro culturing, transfection, selection, as well as transplanting the DNA vector containing-cells to promote stable expression of the heterologous gene of interest.

Methods of presenting the DNA molecule to the target kidney tissue includes, but is not limited to, encapsulation of the DNA molecule into cationic liposomes, subcloning the DNA sequence of interest in a viral or plasmid vector, or the direct injection of the DNA molecule itself into the kidney. The DNA molecule, regardless of the form of presentation to the kidney, is preferably presented as a DNA vector molecule, either as a recombinant viral DNA vector molecule or a recombinant DNA plasmid vector molecule. Expression of the heterologous gene of interest is ensured by inserting a promoter fragment active in eukaryotic cells directly upstream of the coding region of the heterologous gene. One of ordinary skill in the art may utilize known strategies and techniques of vector construction to ensure appropriate levels of expression subsequent to entry of the DNA molecule into the kidney tissue.

Thus, in a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors, or by direct injection of naked DNA, or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors) and so on. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor. Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In another specific embodiment, viral vectors that contain nucleic acid sequences encoding the polypeptide are used. The nucleic acid sequences encoding the polypeptide to be used in gene therapy are cloned into one or more vectors, which facilitate delivery of the gene into a patient. Retroviral vectors, adenoviral vectors and adeno-associated viruses are examples of viral vectors that may be used. Retroviral vectors contain the components necessary for the correct packaging of the viral genome and integration into the host cell DNA.

Adeno-associated virus (AAV) and adenoviruses are especially attractive vehicles for delivering genes. Targets for adenovirus-based delivery systems may include liver, the central nervous system, endothelial cells, respiratory epithelia and muscle. Adenoviruses have the advantage of being capable of infecting non-dividing cells.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. These cells are then delivered to a patient.

In one embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion and so on. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and so on.

In a preferred embodiment, the cell used for gene therapy is autologous to the patient.

Thus, in a preferred embodiment, kidney cells recovered from the kidney are cultured in vitro for subsequent utilization as a delivery system for gene therapy. It will be apparent that the invention is not limited to any specific kidney tissue. It would be possible to utilize any tissue sources for in vitro culture techniques. The method of using the gene of this invention may be employed both prophylactically and in the therapeutic treatment of nephritic-related disease.

A further embodiment of this invention includes the method as hereinbefore described including introducing the gene into the cell in vitro. This method also includes subsequently transplanting the infected cell into the mammalian host. This method includes after effecting the transfecting of the kidney tissue cell but before the transplanting of the infected cell into the mammalian host, storing the transfected kidney tissue cell. It will be appreciated by those skilled in the art that the infected kidney tissue cell may be stored frozen in 10 percent DMSO in liquid nitrogen. This method includes employing a method to substantially prevent the development of nephritis-related diseases in a mammalian host having a high susceptibility of developing such a disease.

Another embodiment of this invention includes a method of introducing at least one gene encoding a product into at least one cell of a kidney tissue of a mammalian host for use in treating the mammalian host as hereinbefore described including effecting in vivo the infection of the cell by introducing the viral vector containing the gene coding for the product directly into the mammalian host. Preferably, this method includes effecting the direct introduction into the mammalian host by intraparenchymal injection or by intravenous injection. This method includes employing the method to substantially prevent a development of nephritis-related disease in a mammalian host having a high susceptibility of developing such a disease. This method also includes employing the method on mammalian host suffering from the nephritis-related disease for therapeutic use.

It will be appreciated by those skilled in the art that the non-viral vectors employing a liposome are not limited by cell division as is required for the retroviruses to effect infection and integration of kidney tissue cells. This method employing non-viral means as hereinbefore described includes employing as the gene a gene capable of encoding a member belonging to an immunosuppressive and/or anti-inflammatory protein and a selectable marker gene, such as an antibiotic resistance gene.

In a specific method disclosed as an example, and not as a limitation to the present invention, a viral vector containing the IL-10 coding sequence was ligated downstream of the CMV promoter.

In a specific embodiment, nucleic acids comprising sequences encoding an immunosuppressive or anti-inflammatory protein or cytokine are administered to treat, inhibit or prevent a disease or disorder associated with nephritis, and in particular, glomerulopathy, and further in particular glomerulosclerosis, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, nucleic acid sequences may encode an immunosuppressive or anti-inflammatory protein or cytokine, in which the nucleic acid sequences are part of expression vectors that express the polypeptides in a suitable host. In particular, such nucleic acid sequences have promoters operably linked to the polypeptide coding region, said promoter being inducible or constitutive, and, optionally, tissue-specific. In another particular embodiment, nucleic acid molecules are used in which the polypeptide coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989).

Therapeutic Composition

In one embodiment, the present invention relates to prevention or treatment for various diseases that are characterized by damage to the glomeruli, which may further result in proteinuria. In this way, the inventive therapeutic composition may be administered to human patients who are either suffering from, or prone to suffer from the disease by providing the gene product that is an anti-inflammatory or immunosuppressant cytokine. In particular, the disease is associated with nephropathology or glomerulonephritis. Further in particular, the present invention is directed to a treatment for glomerulosclerosis.

The pharmaceutical forms suitable for injectable use include sterile aqueous solutions (where water soluble) In all cases the form must be sterile and must be fluid to the extent that easy syringability exists. It must be stable under the conditions of manufacture and storage and must be preserved against the contaminating action of microorganisms such as bacteria and fungi. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of gene therapy such an active material for the treatment of disease in living subjects having a diseased condition in which bodily health is impaired.

Delivery Systems

Various delivery systems are known and can be used to administer a nucleic acid of the invention, e.g., encapsulation in liposomes, surface complexation with cationic liposomes, complex formation with peptide or protein transduction domains, microparticles, microcapsules, recombinant cells capable of expressing the gene product, receptor-mediated endocytosis, construction of a nucleic acid as part of a retroviral or other vector. Administration can be systemic or local. However, local administration is preferred. In addition, it may be desirable to introduce the pharmaceutical compositions of the invention into the kidney by any suitable route, including direct intraparenchymal injection or femoral vein injection. Examples of liquid composition include formulations adapted for injection subcutaneously, intravenously, intraarterially, intrathecally, Examples of aerosol formulation include inhaler formulation for administration to the lungs.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

Effect of Exogenous Cytokine Expression on Glomerulosclerosis

Cytokines such as IL-1, tumor necrosis factor-a (TNF-a) and substances derived from platelets have a role in augmenting neutrophil-mediated glomerular damage (Johnson R J et al., Semin Nephron 11: 276-284 (1991)). Through the release of inflammatory cytokines, macrophages may also stimulate mesangial cells to proliferate and synthesize ECM proteins. The accumulation of ECMs such as collagen, and a-smooth muscle actin is critically related to renal injury and is probably induced by cytokines such as TGF-β, platelet derived growth factor (PDGF), endothelin and angiotensin II (Eddy AA et aL, J Am Soc Nephrol 7: 2495-2508 (1996)). These suggest that the reduction of the disease mediators could be an effective means for the intervention of glomerular damage. To attenuate the pathological consequences of inflammatory responses in the glomerulus, the exogenous introduction of anti-inflammatory or immunosuppressant cytokines or blocking the production of pro-inflammatory cytokines could be beneficial and this approach has been actually attempted for some inflammatory diseases (Kitching AR et al., Kidney Int 52: 52-59 (1997); Rui-Mei L et al., Kidney Int 53: 845-852 (1998); Engelhardt M et al., J Clin Oncol 14: 1405-1406 (1996); Matsumoto K et al., Nephron 73: 305-309 (1996); Gerritsma J S et al., J Am Soc Nephrol 8: 1510-1516 (1997)). Such protein may include without limitation IL-1Ra, IL-4, IL-6, IL-10, IL-16 and TGF-β,

IL-10 produced by monocytes, Th2 and B cells play an important part in the down regulation of major histocompatibility complex (MHC) class II and the inhibition of Th1 cells. In earlier reports, IL-10 has been successfully employed for the suppression of collagen-induced inflammatory arthritis and other inflammatory diseases (Apparailly F et al., J Immunol 160: 5213-5220 (1998); Go NF et al., J Exp Med 172: 1625-1631 (1990); Rogy M A et al., Hum Gene Ther 11: 1731-1741 (2000); Oberholzer C et al., Proc Natl Acad Sci USA 98: 11503-11508 (2001)). In this study, IL-10 exogenously expressed via an adenoviral vector was shown to inhibit FSGS progression effectively.

The mice with exogenous IL-10 expression were found to have a significant reduction of the glomerular sclerotic index at 10 weeks when compared with LacZ treated or naive control. These results indicate that immunosuppression by IL-10 expression in the kidney can prevent the onset of glomeruloclerosis. Ad:hIL-10 transduction was only performed in the left kidney but we observed similar therapeutic effect in the right kidney, as well. This was expected because IL-10 expression was alos detected in the contralateral kidney of the treated mice (Table 1).

TABLE 1
Human IL-10 levels in sera, the kidney, the liver and the spleen of FGS/Kist mice
transduced with 10⁸ p.f.u. of Ad:hIL-10 by parenchymal injection.

| | Human IL-10 (ng/g wet weight) | | | | |
|---|---|---|---|---|---|
| Day | Sera (ng/ml) | Transduced kidney | Contralateral kidney | Liver | Spleen |
| 1 | 14 ± 2 | 4.12 ± 1.33 | 2.46 ± 1.33 | 4.53 ± 1.7 | 12.74 ± 3.7 |
| 5 | 24 ± 4.2 | 7.99 ± 5 | 3.13 ± 1.2 | 5.99 ± 3.33 | 13.47 ± 2.91 |
| 10 | 0.6 ± 0.1 | 2 ± 0.33 | 1.33 ± 1.1 | 1.86 ± 1.33 | 4.73 ± 2.73 |
| 15 | 0.54 ± 0.4 | 13.3 ± 5.3 | 1.73 ± 1.33 | 8.66 ± 4.99 | 4.73 ± 1.82 |
| 20 | 0.14 ± 0.05 | 11.7 ± 5 | ND | 5 ± 9.99 | 3.64 ± 1.87 |

Data are presented as mean ± s.d. values.
ND, not determined.
n = 3 per each group.

Examination of reporter-gene expression in FGS/Kist mice showed that LacZ expression in the left kidney was highest in 24 h post transduction and decreased to a base level in 5 days. This rather short duration of X-gal staining in the transduced tissue can be explained by immune responses to adenoviral vector, expression of the LacZ transgene, and damage to renal tissue by parenchymal injection (Bosch R J et al., Exp Nephrol. 1: 49-54 (1993)). In fact, the transgene expression of LacZ was found to last a much shorter time than IL-10 expression. This is probably caused by the strong immunogenicity of the LacZ protein (Minter RM et al., Proc Natl Acad Sci USA 98: 277-282 (2001)). Presently, efforts are underway to reduce the vector-mediated immune response by employing the adeno-associated virus (AAV) vector. It is also interesting that there was consistent X-gal staining in the cortex of the contralateral kidney, although gene expression was much lower than in the left kidney. This phenomenon may be explained by an earlier report that the cellular trafficking of GFP positive cells via the lymph node was detected in the contralateral joint or by the leakage of circulating adenoviral vectors by excessive tissue damage inflicted upon intraparenchymal injection (Lechman E R et al., J Immunol 163: 2202-2208 (1999)). In fact, the tubular uptake of FITC-oligonucleotides was reported in the contralateral kidney (Tomita N et al., J Am Soc Nephrol 11: 1244-1252 (2000)).

Viral or nonviral vectors for gene therapy as well as genetically modified renal cells have been used for the delivery of foreign genes in the kidney. Various vectors were injected into renal cells through different routes, via intraarterial, intraureteral or intraparenchymal injections (Bosch R J et al., Exp Nephrol 1: 49-54 (1993); Ye X et al., Hum Gene Ther 12: 141-148 (2001)). The major limitation of intraparenchymal injection was that it caused renal injury. The delivery of a transgene to the kidney ex vivo prior to transplantation into a recipient could also be used in some cases. Like other gene therapies, several problems remain. These are the identification of an appropriate target disease and the development of an improved vector that allows prolonged gene expression as well as enhanced efficacy in vivo.

Presently, alleviation of proteinuria and hypertension using immunosuppressive drugs such as steroids and cyclosporin are the methods for treating nephritis. However, long-term exposure to these drugs induces side effects in several organs. Local administration of IL-10 that lasts for a prolonged period may be a logical approach for preventing the progression of glomerulonephritis.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Cell Lines and Animals

Cell line 293 (Microbix, Toronto, Ontario, Canada) was cultured in DMEM medium containing 10% heat-inactivated calf serum (Hyclone, Logan, Utah, USA), penicillin (100 U/ml) and streptomycin (100 μg/ml) in a humidified, 5% CO₂ incubator at 37° C. Male and female FGS/Kist mice were supplied by the Korea Research Institute of Bioscience and Biotechnology (KRIBB, Daejeon, Korea). Water and food were provided ad libitum. After adaptation for 1 week, mice were used for experiments.

### Example 2

### Construction of an Adenoviral Shuttle Vector (pAd-YC2)

pΔ E1sp1A (Microbix) was digested with BgIII, and HindIII. The cytomegalovirus (CMV) promoter was also generated from the digestion of pRC/CMV (Invitrogen, Carlsbad, Calif., USA) with BglII and HindIII. The CMV promoter was ligated into pΔ E1sp1A, resulting in an adenoviral shuttle vector with the CMV promoter and multiple cloning sites, which was termed pAd-YC. pAd-YC was cut with ClaI and blunted to accommodate a synthetic linker with several restriction sites, BamHI, ClaI, NheI and ApaI as well as the bovine growth hormone polyadenylation signal sequence (BGHp(A)). BGHp(A) was derived from pRC/CMV after digestion with ApaI and PvuII. pAd-YC, the synthetic linker and BGHp(A) were triple ligated to form another shuttle vector, pAd-YC2.

### Example 3

### Recombinant Ad Vectors

The type 5 adenoviral vector which was replication defective with the El deletion was constructed to encode the human IL-10 cDNA under the control of the CMV promoter and contained the BGHp(A) sequence. We also constructed a type 5 adenoviral vector harboring the β-galactosidase gene (Ad:LacZ) as a control. For homologous recombination, a shuttle vector, pAd-YC2, and a rescue vector, pJM17 (Graham F L and Van Der Eb A J, Virology 52: 456-467 (1973); Mcgrory M J et aL, Virology 163: 614-617 (1988)), were co-transfected into 293 cells by Tfx-20™ (Promega, Madison, Wis., USA) mediated lipofection according to the manufacturer's protocol. 293 cells were plated 24 h prior to transfection at 70% confluency in a 24-well plate. Thirty microliters of Tfx-20, 4 μg of the shuttle vector, and 6 μtg of pJM17 were used for each plate. After the transfection, cells were fed with fresh DMEM every 3 days until the onset of the cytopathic effect (CPE). To purify pure plaques, cell culture supernatant was serially diluted in serum-free media and incubated with 293 cells at 37° C. for 1 h. The mixture of 1.5 ml of 2× medium plus 1.5 ml of 1% agarose was overlayed on 293 cells. After 7 days, plaques that were well isolated were further purified and propagated in 293 cells and screened by PCR using upstream primers derived from the CMV promoter and downstream primers from the BGHp(A) sequence. Viruses containing the human IL-10 cDNA or the LacZ gene were then amplified and purified in cesium chloride density gradient. Viral titer was determined by agarose overlay. After the CsCl purification, purified viruses were dialyzed in PBS containing 10% glycerol. Viral stocks were stored at −70° C.

### Example 4

### In vivo Administration of Recombinant Ad Vectors

FGS/Kist mice at the age of 6 weeks were anesthetized with ketamine (70 mg/kg) and xylazine (7 mg/kg) by intraperitoneal injections. The left kidney was exposed via a flank incision and injected into the parenchyma with 20 μl of Ad:hIL-10 (5×109 p.f.u./ml) and 20 μl of Ad:LacZ (5×109 p.f.u./ml) using a Hamilton syringe (Hamilton, Reno, Nev. USA).

### Example 5

### In situ RT-PCR to Detect IL-10 Transcript

Mice transduced with Ad:hIL-10 was sacrificed one day after the parenchymal injection. Cryostat sections of the perfusion-fixed kidney were cut to 10 μm thickness and mounted on Poly-PrepTM (Sigma, St, Louis, Mo., USA) slides with the Tissue-TekTM OCT compound (Miles, Elkhart, Ind., USA). The OCT compound was washed out with DEPC-PBS. Sections were delipidated with graded ethanol washes, rehydrated, and permeabilized in 0.1% Triton X-100 for 90 sec. The detergent was washed with DEPC-PBS and the tissue was incubated with 20 μl of proteinase K (1 mg/ml) solution for 10 min and inactivated for 5 min at 95° C. Then, 5 units of RNase-free DNase I (Promega) were added to the tissue, and the tissue was incubated at 37° C. overnight. The next day, slides were incubated at 75° C. to inactivate DNase I activity and were washed with DEPC-PBS. RT-PCR was carried out in 5× reaction buffer (Promega) with 10×DIG labeling Mix (Roche Molecular Biochemicals, Mannheim, Germany), 5 units of AMV reverse transcriptase, 5 units of Tfl polymerase, and 1 mM of MgSO4. Primers corresponded to the coding region of human IL-10 (upstream primer: 5′-cat gca cag ctc agc act gct c-3′ (SEQ ID NO:1), downstream primer: 5′-gtc gcc acc ctg atg tc-3′ (SEQ ID NO:2)). The tissue sections were then washed successively in 2×SSC, 1×SSC, and 0.5×SSC for 10 min each. The sections were blocked in 100 mM maleic acid and 150 mM NaCl containing 1% BSA and 5% calf serum for 30 min. The slides were incubated for 1 h at room temperature with anti-dig alkaline phosphatase conjugated antibody (Roche Molecular Biochemicals) diluted 1000 fold with 0.1% BSA solution. Tissues were then incubated with NBT/BCIP (Sigrna) for 10 min and the reaction was stopped with 0.5 M EDTA. The tissues were dehydrated and mounted with phosphate-glycerol for observation.

### Example 6

### Evaluation of Gene Expression

One day after the intraparenchymal injection of Ad:LacZ, the FGS/Kist mice were sacrificed. The kidneys were harvested and examined for β-galactosidase expression by employing both LacZ staining and colorimetric assay. After the dissection of individual kidneys, kidneys transduced with Ad:LacZ were fixed with 0.05% glutaraldehyde in PBS by perfusion for 15 min, and incubated in X-gal solution (1.5 mM potassium ferricyanide, 1.5 mM potassium ferrocyanide, and 1% X-gal) at 37° C. for 4 h. The kidney sections were then embedded with paraffin. Tissue sections of 4 μm were counterstained with eosin and hematoxylin. Colorimetric assay for βgalactosidase expression was performed as follows: whole kidney tissue was homogenized in ice-cold 0.25 M Tris-HCl (pH 8.0) on days 1, 3, 5 and 10 after transductions. Homogenate was subjected to freezing and thawing 3 times, and was centrifuged at 3000 rpm for 5 min. The cell lysates of 30 μl were incubated with 150 μl of 2×β-galactosidase buffer (200 mM sodium phosphate, pH 7.3, 2 mM MgCl2, 100 mM β-mercaptoethanol, and 1.33 mg/ml ONPG) for 2 h at 37° C. The reaction was stopped by adding 500 μl of 1 M sodium carbonate. β-galactosidase activity in the extract was measured for O. D. at 420 nm and normalized for a total protein content. The protein content was measured with a protein assay kit (Bio-Rad, Hercules, Calif., USA). The assay was calibrated with a standard curve that was generated by using purified β-galactosidase. Gene expression in the kidney was expressed as cellular β-galactosidase activity per mg protein.

### Example 7

### Measurement of IL-10

Serum and homogenated tissue of the kidney, the liver and the spleen were harvested on days 1, 5, 10, 15 and 20, and stored at −70° C. until tested. IL-10 concentration in serum and tissue was quantitated using an enzyme linked immunosorbent assay (ELISA) kit (BioSource International, Camarillo, Calif., USA). In brief, 96-well plates coated with antibodies specific for IL-10 were incubated with serum samples or standards for 3 h at 37° C. Plates were washed, and biotinylated anti IL-10 antibodies were added into each well. After 1 h incubation at room temperature, plates were washed and incubated with streptavidin-horseradish peroxidase conjugates for 1 h at room temperature. After washing, enzyme activity was determined by adding 2, 2-Azino-bis-(3-ethybenzthiazoline-6-sulfonic acid) and measured for optical density at 405 nm in a microplate reader. The means of duplicated wells were subtracted with the means of a control, and IL-10 concentrations were calculated by using a standard curve.

### Example 8

### Proteinuria Assay

Urine samples of FGS/Kist mice transduced with either Adh:IL-10, Ad:LacZ or naive controls were collected 3 weeks after the transductions. Urine was individually collected from 6 mice using micropipettes once a day for one week. Proteinuria was measured with the Lab-stix kit (New England Medical Supply, Providence, R.I., USA). Grading was done on a scale from 0 to 3.

### Example 9

### Histological Analysis

Kidneys from FGS/Kist mice before or after transduction were fixed in 10% buffered-formalin and were paraffin-embedded. Sections of 4 μm thickness were mounted on slides and stained with periodic acid-schiff (PAS). To quantify renal tissue damages, areas occupied by glomerulosclerosis were calculated using a microscope. The glomerular sclerotic scores were determined in a range from 0 to 2 (0=normal, 1<50%, 2>50%) according to the severity o glomerulus. The glomerular sclerotic index =(number of glomeruli with severity 1)+(number of glomeruli with severity 2×2)/total number of glomeruli.

### Example 10

### Quantitative RT-PCR

Four weeks after Ad:LacZ or Ad:hIL-10 transduction, total RNA was purified from the kidney using the Tri-PureTM (Roche Molecular Biochemicals) reagent. Transcript levels of both TGF-β1 and GAPDH were measured using real-time quantitative PCR (MJ Research, San Francisco, Calif., USA). Primer sequences were as follows: TGF-β1: 5′-gac tac tat gct aaa gag gtc acc-3′(sense, SEQ ID NO:3) and 5′-ctg atc ccg ttg att t cc acg tgg-3′ (antisense, SEQ ID NO:4); GAPDH: 5′-cca gta tga ctc cac tca cgg-3′ (sense, SEQ ID NO:5) and 5′-caa cct ggt cct cag tgt agc c-3′ (antisense, SEQ ID NO:6). Results were expressed as the ratio between amplified DNA fragments of TGF-β1 and GAPDH mRNA respectively.

### Example 11

### Immunostaining for TGF-β

Cryostat sections were incubated in sequence in Bouin's fixative for 5 min, in acetone at 4° C. for 10 min, in methanol at −20° C. for 15 min, in 2% paraformaldehyde for 2 min, in 4% paraformaldehyde for 4 min, and in 70% ethanol for 10 min. The sections were then rehydrated with graded ethanol, and were washed with PBS and incubated with methanol containing 0.3% H2O2 to remove endogenous peroxidase activity for 30 min. Blocking was done in PBS containing 10% FBS for 1 h and incubated overnight with a rabbit anti-TGF-β1 antibody (Santa Cruz Biotechnology, Santa Cruz, Calif., USA) in PBS containing 0.5% BSA and 2% FCS at 4° C. The tissues were incubated with anti-rabbit HRP conjugates (Sigma) for 1 h at room temperature. The tissues were then incubated with diamine benzidine (Sigma) for 5 min, dehydrated and mounted with phosphate-glycerol for observation.

### Example 12

### Statistical Analysis

Results are expressed as means ± standard deviation (s.d.). Statistical significance was determined using Student's t test. P<0.05 was considered significant.

### Example 13

### Efficient Transduction of Recombinant Adenoviruses in the Renal Tissue

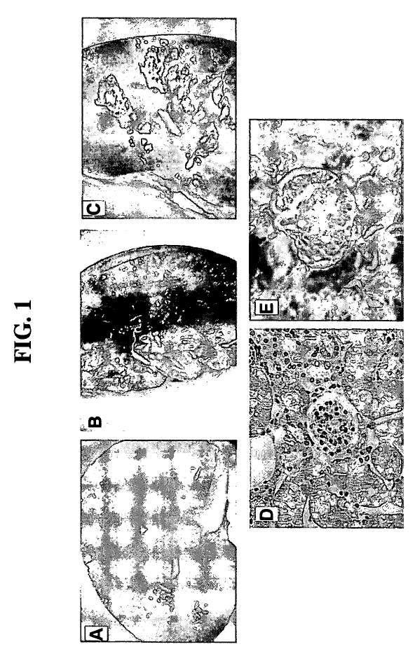

To examine transduction efficiency of recombinant adenoviruses in the kidney, Ad:LacZ was injected into the kidney. Transduction efficiency was determined by X-gal staining of kidney sections embedded with paraffin. Twenty microliters of Ad:LacZ (5×10⁹ plaque forming units (p.f.u.)/ml) were injected into the parenchyma of the left kidney that was exposed by a flank incision of the mice. Twenty four hours after the injection of Ad:LacZ into the kidney, both the right and the left kidneys were harvested, fixed by glutaraldehyde perfusion, and soaked in X-gal solution at 37° C. for 4 h. The injection resulted in significant transduction of the cortex and medulla of the left kidney compared with the kidney of sham control mice (FIG. 1A and 1B). Interestingly, β-galactosidase activity was also detected in the contralateral (right) kidney, at a lower protein level than that of the injected kidney (FIG. 1C). X-gal staining was detected in the cortex but not in the medulla of the right kidney. Microscopic examinations of sections of the left kidney exhibited X-gal staining in the Bowman's capsular epithelium as well as the tubular epithelium (FIG. 1E). However, we could not detect broad staining of glomeruli and interstitial cells. These results indicate that, after parenchymal injection, recombinant adenoviruses can efficiently transduce some types of renal cells but not all cell types.

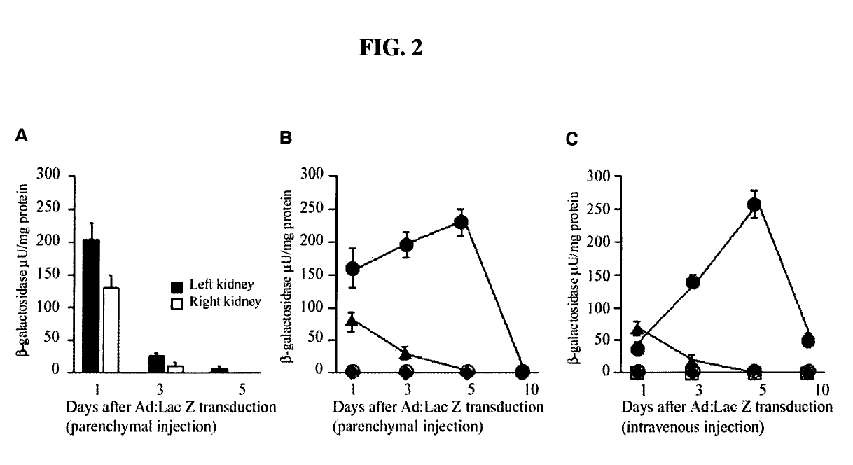

Next, we examined the levels of LacZ expression on days 1, 3 and 5 in both the left and the right kidneys. LacZ expression in the left kidney was 204 μU±24 per mg protein on day 1, 18.1 μU±2.5 on day 3 and 2.3 μU±0.6 on day 5 (FIG. 2A). There was also LacZ expression in the contralateral kidney, and LacZ expression was 131.9 μU±23 per mg protein on day 1 and 9.2 μU ±3.4 on day 3. LacZ expression was also examined in other tissues of the transduced mice to detect the leakage of locally injected viruses. LacZ expression was not detected in the heart and the lung, but a high level of LacZ expression was found in the liver for 5 days (168.5 μU±29 per mg protein on day 1). A relatively low level of LacZ expression was also detected in the spleen for 3 days (77.52 μU±14 per mg protein on day 1) (FIG. 2B). However, LacZ expression in the kidney was not detected upon intravenous injection of Ad:LacZ. In both experimental groups, peak expression was seen on day 1 in the spleen and on day 5 in the liver (FIG. 2C).

### Example 14

### Efficient Transduction of Ad:hIL-10 in vivo

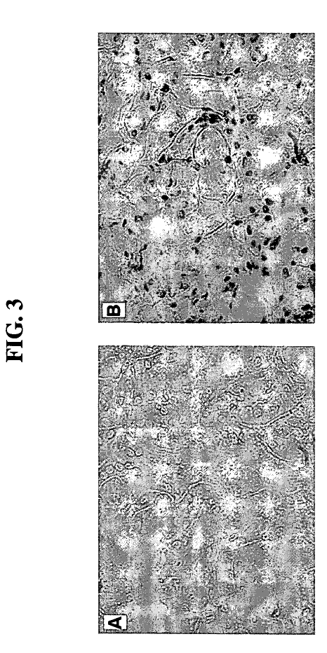

As Ad:LacZ exhibited efficient transduction of the kidney tissue, especially of Bowman's capsular epithelia and tubular epithelia, we examined if Ad:hIL-10 transduces the tissue and is efficiently expressed in vivo. In situ RT-PCR was performed to determine Ad:hIL-10 expression in the renal tissue one day after injection. Brown staining, which indicates the presence of IL-10 transcript, was shown in the parenchymal tissue of the kidney transduced with Ad:hIL-10 but not with Ad:LacZ (FIG. 3). To confirm human IL-10 production in mice, ELISA was performed using sera, the kidney, the liver and the spleen after transduction. Sera and each organ from mice transduced with 20 μl of Ad:hIL-10 (5×10⁹ p.f.u./ml) were harvested on days 1, 5, 10, 15 and 20. IL-10 levels in the kidney peaked biphasically on days 5 and 15, respectively after the parenchymal injection and were sustained over 20 days (Table 1). IL-10 was detected in the liver in a similar biphasic manner, though slightly lower than in the kidney. In the spleen, however, the peak level of IL-10 was found on day 5 and decreased gradually. Interestingly, IL-10 levels were also detected for 15 days in contralateral kidneys, and the serum level of IL-10 peaked on day 5 and declined rapidly afterward.

### Example 15

### The Inhibition of FSGS by Ad:hIL-10 Transduction

In the FGS/Kist mouse strain, there is a spontaneous development of glomerular damage, which is characterized by FSGS. Proteinuria is also detected in the mice from 6 weeks of age. At the age of 6 weeks, glomerular sclerotic index of male and female was 0.05±0.03 and 0.06±0.06, respectively. But, at the age of 10 weeks, glomerular sclerotic index was 0.69±0.32 and 0.12±0.08 in male and female mice, respectively (Table 2). The sclerotic index was found to be markedly increased in male mice, and somewhat less but still significantly increased in female mice. This was consistent with the natural development of glomerular damage in this type of mouse.

Six mice from each group were transduced with 10⁸ p.f.u. of Ad:hIL-10 or Ad:LacZ at the age of 6 weeks. The mice were sacrificed at 6 weeks (naive control I) or at 10 weeks (naive control II, Ad:LacZ, and Ad:hIL-10). The glomerular sclerotic index was scored. Values shown represent the mean±s.d. values of kidneys from six mice after viral transduction. The glomerular sclerotic scores were determined in a range from 0 to 2 (0: normal; 1:<50%; 2:>50%) according to the severity of glomeruli.

TABLE 2
Reduction of glomerular sclerotic index in FGS/Kist
mice transduced with Ad:hIL-10.

Glomerular sclerosis Index^{a}

| Group/Age (Weeks) | MALE | FEMALE |
|---|---|---|
| Naive control I (6) | 0.05 ± 0.03 | 0.06 ± 0.06 |
| Naive control II (10) | 0.69 ± 0.32 | 0.12 ± 0.08 |
| Ad:LacZ (10) | 0.54 ± 0.43 | 0.10 ± 0.06 |
| Ad:hIL-10 (10) | 0.01 ± 0.02* | Negligible** |
| ^{a}A total intensity score was calculated for each specimen according to the following equation: | | |
| # Glomerular sclerotic index = (number of glomeruli with severity 1) + (number of glomeruli with | | |
| # severity 2 × 2) / total number of glomeruli. | | |

*P < 0.001 in the male group treated with Ad:hIL-10 versus male control II.
**P < 0.01 in the female group treated with Ad:hIL-10 versus female control II.
These results are representative of three separate experiments.

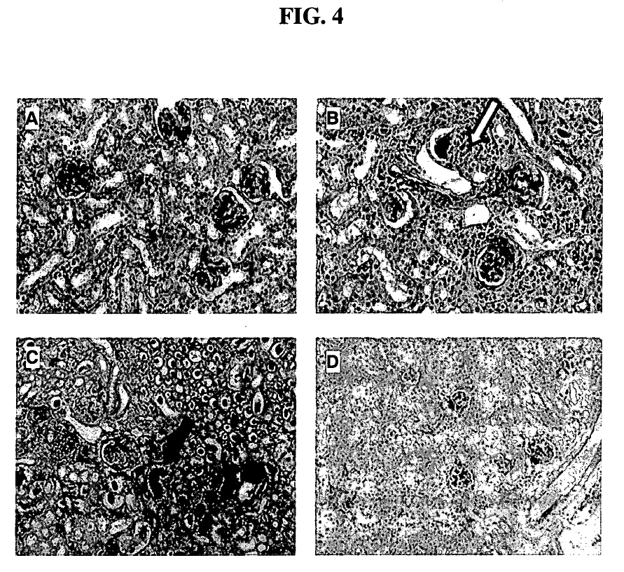

In FSGS, the infiltration of inflammatory cells and subsequent tissue inflammation play a key role in disease progression. As IL-10 is a potent immunosuppressive cytokine, exogenous IL-10 could be effective in the inhibition of FSGS progression. Ad:hIL-10 was found to transduce the kidney efficiently and IL-10 was expressed at a high level as shown above, although the expression was transient due to the nature of adenoviral vectors. We then analyzed the efficacy of IL-10 in the inhibition of glomerulosclerosis in the kidney of FGS/Kist mice. The mice were transduced with Ad:hIL-10, or Ad:LacZ at 1×108 p.f.u. in the left kidney at the age of 6 weeks. At that time, FSG/Kist mice did not show detectable histological damages of glomeruli and tubules of the kidney. However, at the age of 10 weeks, tubular atrophy, the infiltration of inflammatory cells, and the fibrosis of interstitium were all evident in the kidney. The areas of kidney abnormalities reached 20% in the kidney cortex of the mice. When mice were transduced with Ad:hIL-10, they were shown to have significantly reduced glomerulosclerosis. The glomerular sclerotic index was 0.01±0.02 (P<0.001) in male mice and was almost zero (P<0.01) in female mice (Table 2). Inflammatory cells were not detected in the kidney of either male or female FSG/Kist mice transduced with Ad:hIL-10. In contrast, mice treated with Ad:LacZ revealed severe glomerulosclerosis and heavy infiltration of inflammatory cells in the kidney (FIG. 4).

### Example 16

### The reduction of Proteinuria by Ad:hIL-10 Transduction

Mice with FSGS often reveal excessive secretion of proteins in urine because of the compromised filtration and/or reabsorption of proteins in glomeruli and in collecting tubules of the kidney. Therefore, we examined if IL-10 expression in the kidney can also be of use in stemming proteinuria. The naive group was shown to start proteinuria at about 6 weeks of age, and severe proteinuria was observed at 10 weeks. Whereas the Ad:LacZ transduced group had a severity level of 3 in about 50% of the urine samples, the mice transduced with Ad:hIL-10 exhibited significantly reduced levels of proteinuria (severity 0 or 1) at 10 weeks (Table 3). These results indicate that IL-10 can effectively inhibit the progression of both FSGS and proteinuria in FGS/Kist mice.

TABLE 3
Reduction of proteinuria in FGS/Kist mice transduced with Ad:hIL-10.

| | Proteinuria (%)^{a} | | | | | | | |
|---|---|---|---|---|---|---|---|---|
| Group/Age | Male (severity scale) | | | | Female (severity scale) | | | |
| (Weeks) | 0 | 1 | 2 | 3 | 0 | 1 | 2 | 3 |
| Naive controlI | 35.7 | 40.4 | 23.9 | 0 | 40.4 | 42.9 | 16.7 | 0 |
| (6) | | | | | | | | |
| Naive controlII | 0 | 21.4 | 28.6 | 50.0 | 2.4 | 11.9 | 38.1 | 47.6 |
| (10) | | | | | | | | |
| Ad:LacZ (10) | 0 | 14.3 | 35.7 | 50.0 | 2.4 | 11.9 | 35.7 | 50.0 |
| Ad:hIL-10 (10) | 42.9 | 52.4 | 4.7 | 0 | 50.0 | 50.0 | 0 | 0 |
| ^{a}Urine was individually collected from each group of 6 mice using micropipettes once a day for 7 days after injection of Ad:hIL-10 (3 weeks after Ad transduction). Proteinuria was measured with the Lab-stix kit. The scale of severity was graded from 0 to 3, and the percent of proteinuria was expressed as (total number of urine samples of each scale) × 100/42 (=total number of test | | | | | | | | |
| # samples). These results are representative of three separate experiments. | | | | | | | | |

### Example 17

### The Suppression of TGF-β1 Expression by Ad:hIL-10 Transduction

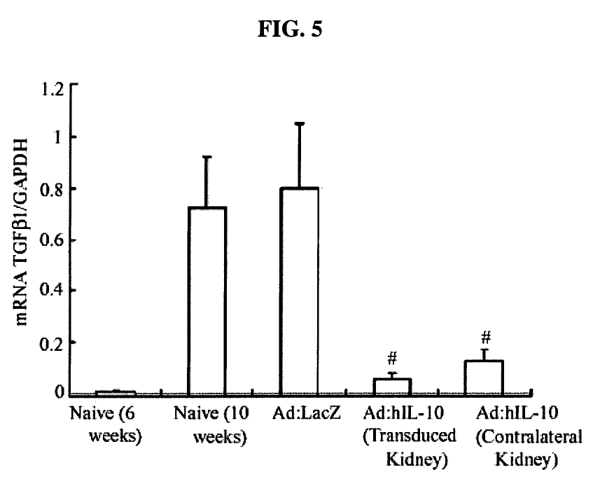

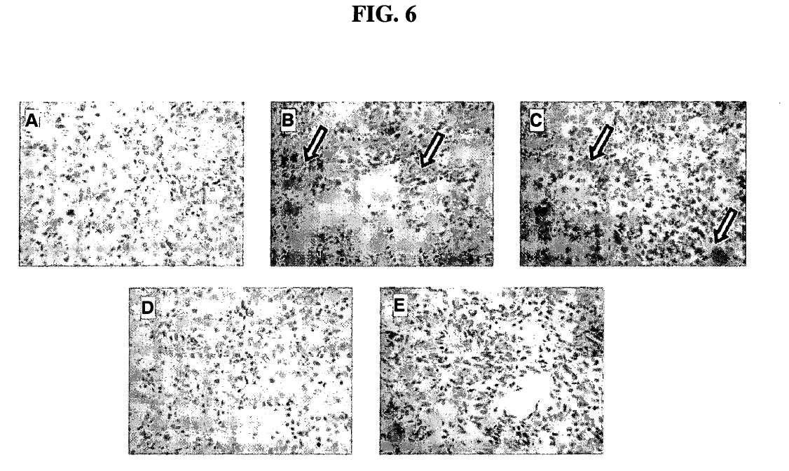

TGF-β1 promotes the synthesis of extracellular matrix (ECM) proteins, leading to the thickening of basement membranes and subsequent glomerulosclerosis. Next, we examined if Ad:hIL-10 reduces TGF-β1 mRNA as well as its proteins in the kidney of FGS/Kist mice. Kidneys were obtained from 6 mice in each group at 10 weeks. Quantification of TGF-β1 mRNA showed that the kidneys treated with Ad:hIL-10 significantly reduced TGF-β1 mRNA (10.2 fold decrease compared with the naive control at 10 weeks; P<0.01) as well as contralateral kidneys (6 fold decrease compared with the naive control at 10 weeks; P<0.01) (FIG. 5). Immunohistochemistry was carried out to determine whether TGF-β1 expression was reduced by Ad:hIL-10 transduction. TGF-β1-specific immunostaining in the kidneys of Ad:hIL-10 treated mice was found to be significantly weak at 10 weeks than in the control groups treated with Ad:LacZ or untreated (FIG. 6).

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of reducing inflammation in kidney of a subject, comprising delivering to the kidney of the subject in need thereof a therapeutically effective amount of a gene encoding an anti-inflammatory or immunosuppressant protein.

2. The method according to claim 1, wherein the gene encodes IL-1Ra, IL-4, IL-6, IL-10, IL-16, or TGF-β1.

3. The method according to claim 2, wherein the gene encodes IL-10.

4. The method according to claim 1, wherein the gene is inserted into a vector.

5. The method according to claim 4, wherein the vector is a virus.

6. The method according to claim 5, wherein the virus is an adenovirus or an adeno-associated virus or retrovirus.

7. The method according to claim 4, wherein the vector is a plasmid.

8. The method according to claim 1, wherein the gene is transfected into a population of cells in vitro, wherein the transfected population of cells is administered to the subject.

9. The method according to claim 8, wherein the gene encodes IL-1Ra, IL-4, IL-6, IL-10, IL-16, or TGF-β1.

10. The method according to claim 9, wherein the gene encodes IL-10.

11. A method of treating nephritis comprising delivering to the kidney of the subject in need thereof a therapeutically effective amount of a gene encoding an anti-inflammatory or immunosuppressant protein.

12. The method according to claim 11, wherein the nephritis is glomerulopathy.

13. The method according to claim 12, wherein the glomerulopathy is glomerulosclerosis.

14. A method of preventing nephritis in a patient predisposed to such a condition, comprising delivering to the kidney of the subject in need thereof a therapeutically effective amount of a gene encoding an anti-inflammatory or immunosuppressant protein.

15. The method according to claim 14, wherein the nephritis is glomerulosclerosis.

16. The method according to claim 14, wherein the gene encodes IL-1Ra, IL-4, IL-6, IL-10, IL-16, or TGF-β1.

17. The method according to claim 16, wherein the gene encodes IL-10.

18. A method of reducing excretion of polypeptides in urine of a subject suffering from a renal disorder comprising delivering to the kidney of the subject in need thereof a therapeutically effective amount of a gene encoding an anti-inflammatory or immunosuppressant protein.

19. The method according to claim 18, wherein the gene encodes IL-1Ra, IL-4, IL-6, IL-10, IL-16, or TGF-β1.

20. The method according to claim 19, wherein the gene encodes IL-10.
